(19) United States
(12) Patent Application Publication
(10) Pub. No.: US 2001/0008935 A1
(43) Pub. Date: Jul. 19, 2001

(54) Novel family of compositions based on alkylpolyglycosides and fatty diols, which are especially useful for the preparation of emulsions which are stable and which possess a good opaque-making effect

(76) Inventors: Alain Milius, Nice (FR); Jean-Pierre Boiteux, Saix (FR); Herve Rolland, Castres (FR); Guy Tabacchi, Castres (FR); Chantal Amalric, Blan (FR)

Correspondence Address: Timothy J. Keefer, Wildman, Harrold, Allen & Dixon, 225 West Wacker Drive, Chicago, IL, 60606, US

(21) Appl. No.: 09/758,698

(22) Filed: Jan. 11, 2001

(30) Foreign Application Priority Data
Jan. 12, 2000 (FR) 0000319

(51) Int. Cl. C07H 15/02

(52) U.S. Cl. 536/18.6

(57) ABSTRACT

The present invention relates to a novel family of compositions based on alkylpolyglycosides and fatty diols, which are especially useful for the preparation of emulsions which are stable and which possess a good opaque-making effect. According to the invention, these compositions comprise: 5 to 95 parts by weight of a mixture of alkylpolyglycosides which is constituted of the products of a reaction of a saccharide and 1,12-octadecanediol; 95 to 5 parts by weight of 1,12-octadecanediol.

The present invention relates to a novel family of compositions based on alkylpolyglycosides and fatty diols, which are especially useful for the preparation of emulsions which are stable and which possess a good opaque-making effect.

The invention finds application especially in the field of cosmetics, in particular for the preparation of foaming formulations.

Alkylglycosides or alkylpolyglycosides (APGs) are well-known non-ionic surfactant compounds which can be used alone, or in combination with other surfactants, in a wide range of industrial applications and especially in the field of cosmetics.

Alkylpolyglycosides have first of all been used as foaming agents and in this application, those the alkyl chain of which comprises from 8 to 14 carbon atoms have proved to be particularly interesting.

More recently, alkylpolyglycosides have been used as emulsifiers, and in this application, those the alkyl chain of which comprises 16 to 18 carbon atoms have proved to be particularly interesting.

The patent application WO 92/06778, in the name of the Applicant, describes, for the first time, the use of mixtures of alkylpolyglycosides and fatty alcohols as self-emulsifying agents.

The term <<self-emulsifying>> designates any agent or composition which is capable of forming a stable emulsion with an aqueous phase, practically without the provision of energy, for example by dispersion in the aqueous phase by slow mechanical agitation.

More specifically, the mixtures described in this prior art document comprise

from 60 to 90% by weight of at least one fatty alcohol having 12 to 22 carbon atoms, and preferably 16 to 18 carbon atoms; and

from 10 to 40% by weight of an alkyl polyglycoside, the alkyl part of which is preferably identical to that of the fatty alcohol.

The self-emulsifiable compositions described in the above-mentioned application are marketed under the designation Montanov® 68 and comprise a mixture of alkylpolyglycosides the fatty chains of which comprise 16 and 18 carbon atoms, as well as a mixture of fatty alcohols of the same fatty chain length.

Furthermore, WO 95/13863, in the name of the Applicant, describes compositions which are also based on alkylpolyglycosides and fatty alcohols, and which are presented in the form of concentrates which are especially useful for the preparation of fluid emulsions.

These compositions are essentially characterised by the fact that they comprise a mixture of at least two alkylpolyglycosides which differ by the nature of their alkyl part.

It is specified that at least one of these alkylpolyglycosides comprises an alkyl chain having from 16 to 22 carbon atoms, and preferably from 16 to 18 carbon atoms.

It is specified further that the alkylpolyglycosides comprising an alkyl part having from 16 to 22 carbon atoms must represent at least 25% of the mixture of alkylpolyglycosides, and in all the Examples, these alkylpolyglycosides comprise 16 or 18 carbon atoms and represent at least 50% by weight of the mixture of alkylpolyglycosides.

It was noted that the compositions based on alkylpolyglycosides and fatty alcohols of the state of the art do not enable emulsions to be obtained which be simultaneously stable and opaque, especially when they are used for the preparation of foaming formulations based on anionic surfactants.

Under these circumstances, the aim of the present invention is to solve the technical problem consisting in providing novel compositions which enable emulsions to be prepared the properties of opacity of which are significantly improved with respect to those of the emulsions obtained from compositions described in the state of the art the content of which has been mentioned above, without a notable loss in stability.

The solution in accordance with the present invention for solving this technical problem consists of novel compositions based on alkylpolyglycosides and fatty diols, characterised in that they comprise:

5 to 95 parts by weight of a mixture of alkylpolyglycosides which is constituted of the products of a reaction of a saccharide and 1,12-octadecanediol;

95 to 5 parts by weight of 1,12-octadecanediol.

Such compositions based on alkylpolyglycosides and fatty diols thus differ essentially from the compositions of the state of the art by the fact that they necessarily comprise alkylpolyglycosides derived from 1,12-octadecanediol and that the monoalcohol is replaced by a diol.

It has been discovered, entirely surprisingly and unexpectedly, that such compositions enable emulsions to be obtained, which have remarkable and particularly interesting properties of opacity and stability, for their uses in the field of cosmetics, particularly within foaming formulations.

The compositions which are preferred within the framework of the present invention are the compositions which comprise:

5 to 60 parts by weight of the above-mentioned mixture of alkylpolyglycosides ; and

95 to 40 parts by weight of 1,12-octadecanediol.

The mixture of alkylpolyglycosides constituted of the product of reaction of a saccharide and 1,12-octadecanediol is in fact constituted of a mixture of any proportion of hydroxyoctadecylpolyglycosides (product resulting from the acetalisation of one of the two hydroxyl groups of the diol, preferably the group which is present in position 1), and of polyglycosyloctadecylpolyglycosides (product resulting from the etherification of the two hydroxyl groups of the diol).

These alkylpolyglycosides can be represented, respectively, by the following formulae I and II:

HO—R—O(G)ₙ (I)

(G)ₘ—OR—O—(G)ₚ (II)

in which:

G represents a saccharide residue

R represents a 1,12-disubstituted octadecane group

n, m and p represent the average degree of polymerisation of each saccharide residue.

The above-mentioned alkylpolyglycosides can comprise a glucose or dextrose, fructose, galactose, mannose, ribose, xylose residue, preferably a glucose residue, as saccharide residue.

It is to be further noted that each unit of the polyoside part of the alkylpolyglycosides mentioned above can be in α or β anomeric form, and the saccharide residue can be of furanoside or pyranoside type.

The average degree of polymerisation of each saccharide residue is generally between 1.05 and 2.5, more preferably between 1.1 and 2.

The expression <<alkylpolyglycoside>> used within the context of the present application therefore designates equally an alkylmonooside (degree of polymerisation equal to 1) or an alkylpolyglycoside (degree of polymerisation greater than 1).

1,12-octadecanediol used for the preparation of the compositions in accordance with the present invention is a diol originating from the hydrogenation of the fatty acids of castor oil.

It is notably marketed by the company HENKEL under the designation SPEZIOL® C 18/2.

This compound, due to its natural origin, can contain minor quantities of impurities. Such impurities can be present in amounts ranging up to 25% by weight of the total weight of diol.

Consequently, the compositions in accordance with the present invention can comprise, in corresponding minor proportions, such impurities or the products of reaction of these impurities with a saccharide.

The composition based on alkylpolyglycosides and fatty diols in accordance with the present invention can be prepared by simple mixing of their constituents in pre-determined proportions desired.

On an industrial scale, they will preferably be prepared according to one of the two routes classically used for the synthesis of alkylpolyglycosides, and for example, by reaction in acid medium between 1,12-octadecanediol and a saccharide having an anomeric OH, such as glucose or dextrose.

Such synthetic routes are well known and have been described in numerous documents, and in particular in the documents of the Applicant mentioned above.

If need be, it will be possible for this synthesis to be completed by operations of neutralisation, filtration, distillation or partial extraction of excess fatty diol, or a discoloration operation.

The compositions based on alkylpolyglycosides and fatty diols in accordance with the present invention can be used for the preparation of pharmaceutical, cosmetic, hygiene or detergent emulsions, in particular foaming emulsions.

Shampoos, liquid soaps, shower gels, foam baths, face make-up remover gels, as well as detergent compositions notably such as washing-up detergents and liquid soaps will be cited in particular amongst the foaming emulsions which can be prepared within the context of the present invention.

In general, such a composition will comprise from 1 to 25% by weight, preferably from 1 to 10%, and more preferably from 1 to 5% by weight of the composition based on alkylpolyglycosides and fatty diols in accordance with the present invention.

These emulsions will further comprise surfactants, notably foaming surfactants.

The following can be cited as foaming surfactants:

amphoteric surfactants, notably betaine and its derivatives such as alkylbetaines, such as N-alkylbetaines or C-alkylbetaines, N-alkylaminobetaines, alkylamidobetaines, alkylphosphoamidobetaines or alkylphosphobetaines, sultaine and its derivatives, such as alkylamidosulphobetaines, imidazoline derivatives, N-alkylglycines, N-alkyl-β-alanines, alkylpolyaminecarboxylates and N-alkylaminobutyrates; the preferred amphoteric surfactants are selected from cocamidobetaine, cocobetaine, cocoamidosultaine, cocoamidopropylbetaine, stearylbetaine, stearyl-amphocarboxylic glycinate, cocoamidopropyl-hydroxysulphobetaine, cocoamphocarboxyglycinate, coco-imidazoline carboxylate;

non-ionic surfactants, notably copra amides, polysorbates, hydrogenated and polyoxyethylenated (OE) and/or polyoxypropylenated (OP) castor oils, OE and/or OP alkylphenols or alkylglycosides the alkyl chain of which comprises 8 to 14 carbon atoms, notably those described in the patent application EP 70 074.

cationic surfactants, notably those of the quaternary ammonium or amine oxides type.

and, advantageously, anionic surfactants, notably such as carboxylates, sulphonates and, above all, sulphates. Alkylsulphates and alkylether sulphates neutralised by a cation, such as sodium, potassium, magnesium or ammonium, are preferred amongst the latter.

Furthermore, it will be possible for these emulsions to comprise an oily phase.

This oily phase can be constituted by another oil classically used, such as, for example, an oil of plant, natural, mineral or synthetic origin.

In general, the emulsions mentioned above will comprise up to 50%, and preferably up to 30% by weight of oily phase as defined above.

These emulsions can be prepared by simple dispersion of the composition in accordance with the invention, and optionally of one or more oils in a hydrophilic phase, in general water or a hydrophilic solvent such as glycerol or sorbitol.

Thus, according to a second aspect, the present application aims to cover emulsions which comprise at least one aqueous phase and an oily phase and, as emulsifier, a composition based on alkylpolyglycosides and fatty diols as defined above.

The emulsions which are particularly preferred within the context of the present invention will generally comprise:

from 1 to 10% by weight of a composition based on alkylpolyglycosides and fatty diols as defined above;

from 0 to 30% by weight and preferably from 2 to 30% by weight of at least one foaming surfactant;

from 0 to 50% by weight of oil; and

an aqueous phase.

The invention will be illustrated in detail by the following Examples, which are given solely as an illustration.

### EXAMPLE 1

Method of preparing a composition in accordance with the invention by direct acetalisation.

226.7 g of 1,12-octadecanediol (marketed by the company Sidobre-Sinnova under the designation SPEZIOL® C18/2) are introduced into a two-liter glass reactor equipped with an efficient mechanical stirrer and a coolant circulation in the double envelope and equipped with a condenser and a temperature probe.

After melting at 90° C. of the SPEZIOL® C18/2 in the reactor over 30 minutes, 22.5 g of anhydrous dextrose are dispersed in the reaction medium and are homogenised at 90° C. for 15 minutes. The temperature is then fixed at 105° C., a value at which 2.5% of 96% sulphuric acid are added.

The reaction medium is then maintained for 6 hours at 105° C. under partial vacuum.

After cooling to 90° C., 2.6 g of a 40% solution of sodium hydroxide are added progressively so as to obtain a pH value of a 5% solution of the reaction medium of between 5.5 and 7.5.

The resulting product is thus drained after sieving on a 250 μm sieve. The solid obtained, white in colour, thus has a melting point of 74.7° C., and a diol content estimated at 89.9%.

### EXAMPLE 2

Method of preparing a composition in accordance with the invention by transetherification.

2a) Preparation of butylglucoside

888 g of butyl alcohol and 50 g of heptane are introduced into a two-liter glass reactor, equipped with a double envelope, an efficient mechanical stirrer, a thermometric probe and a Dean Stark type distillation system.

The medium is brought to 85° C., a temperature at which 360 g of anhydrous dextrose and 1.8 g of 96% sulphuric acid are added.

The mixture is then heated at 105-110° C. for 4 hours with continuous recirculation of the heptane/butanol mixture which distils.

2b) Transetherification

624.9 g of the preceding reaction medium, containing 65 to 60% of residual butanol are heated to 85° C. and 642.2 g of 1,12-octadecanediol (SPEZIOL® C18/2) are added in dispersion.

The mixture is then heated at 95° C. under partial vacuum for 5 hours with continuous distillation of the butyl alcohol.

The reaction medium is then cooled to 85° C., neutralised so as to obtain a pH value of a 5% solution of the reaction medium of between 5.5 and 7.5 with a sodium hydroxide solution.

The product obtained thus contains 50% of diol.

### EXAMPLE 3

Method of preparing a composition in accordance with the invention by direct acetalisation

816.3 g of 1,12-octadecanediol (marketed by the company Sidobre-Sinnova under the designation Speziol® C_{18/2}) are introduced into a two-liter glass reactor, equipped with an efficient mechanical stirring and a coolant circulation in the double envelope and equipped with a condenser and a temperature probe.

After melting at 95° C. of the Speziol® C_{18/2}, 198.0 g of xylose are progressively dispersed in the reaction medium and are homogenised at 95° C. for an hour.

3.03 g of 96% sulphuric acid and 2.01 g of 50% hypophosphorous acid are added and the reaction mixture is maintained at 95° C. for 4 hours under partial vacuum, with bubbling of nitrogen.

After cooling to 80° C., the mixture is neutralised so as to attain a pH value of the reaction medium of around 7.2 by adding a solution of sodium borohydride in sodium hydroxide solution.

The resulting product is thus drained after sieving on a 250 μm sieve. The solid obtained, of beige colour, thus has a diol content estimated at 55%.

### EXAMPLE 4

Demonstration of the opaque-making properties of the emulsions obtained by making use of the compositions according to the invention, by comparison to the compositions of the state of the art.

Various emulsions were prepared by means of the compositions of Examples 1 and 2 according to the invention, as well as compositions of the state of the art.

These emulsions were prepared in the following manner:

The compositions based on APG and fatty alcohols are melted in the hot in an aqueous solution of anionic surfactants. The whole is homogenised with stirring and is diluted in the remaining water and then cooled.

In following the experimental method, various emulsions were prepared from the compositions according to the invention described in Examples 1 and 2 and from comparative compositions described in Examples 1 of the document WO 92/06778 (Comparative Example 1) and 1 of the document WO 95/13863 (Comparative Example 2), respectively.

These emulsions have the following compositions:

| | |
|---|---|
| compositions according to the invention | 5%, |
| or according to a Comparative Example: | |
| anionic surfactant (LESNA): | 10%, |
| water: | 85%. |

The stability of the emulsions thus prepared is verified by ageing at 40° C.

The opacity is measured with the aid of a MINOLTA CR 200 chromameter equipped with a cone and a protection tube CR-A33a.

The measurements are made by immersing the cone into 50 ml of emulsion.

The opacity is described by the parameter L, varying from 0 to 100.

The results obtained were reported in Table I below:

In this Table:

Dph signifies: dephasing after

D signifies: days

TABLE I

| | |
|---|---|
| EXAMPLES | |
| ACCORDING TO | COMPARATIVE |
| THE INVENTION | EXAMPLES |

| | 1 | 2 | 1 | 2 |
|---|---|---|---|---|
| Stability | >D7 | >D7 | dph D7 | dph D1 |
| L value | 78.3 | 80.1 | 72 | 30.8 |

As shown in Table I, the emulsions obtained from the compositions of the invention have an opaque-making effect which is significantly greater than that of the emulsions obtained from the compositions of the state of the art, with increase in stability.

### EXAMPLE 5

Examples of emulsions incorporating the compositions based on alkylpolyglycosides and fatty diols according to the invention

LIQUID SOAP

| | | |
|---|---|---|
| A) | Composition of Example 1 | 4% |
| | Water | 30% |
| B) | Les Na | 5% |
| | ORAMIX ® NS 10 | 3% |
| | Water | qs |
| C) | CAPIGEL ® 98 | 2% |
| | Water | qs |
| D) | Perfume | qs |
| | Preservative | qs |

The composition of Example 1 melted in water at about 80° C. (phase A) with stirring. After cooling to around 30° C., phases B, C and D were added. The pH was adjusted to around 7 with sodium hydroxide.

FOAMING DE-MAKE-UP MILK

| | | |
|---|---|---|
| A) | Composition of Example 2 | 4% |
| | Jojoba oil | 4% |
| B) | Water | qs |
| | CAPIGEL ® 98 | 3% |
| C) | Perfume preservative | qs |
| | LESNA | 7% |

Phase A is melted around 80° C. Phase B is added with stirring to phase A thus melted, cooling is done to around 30° C. and then phase C is added and the pH is adjusted to around 7.

DE-MAKE-UP MILK

| | | |
|---|---|---|
| A) | Composition of Example 2 | 4% |
| | Liquid paraffin | 15% |
| B) | Water | |
| C) | SEPIGEL ® 305 | 1% |
| D) | Perfume, preservative | |

The composition of Example 2 is melted in the oil at around 80° C. Phase B is added with stirring to phase A thus melted. Phase C is added at around 60° C., and then phase D is added at around 30° C.

What is claimed is:

1. Compositions based on alkylpolyglycosides and fatty diols, which comprise:
5 to 95 parts by weight of a mixture of alkylpolyglycosides which is constituted of the products of a reaction of a saccharide and 1,12-octadecanediol;
95 to 5 parts by weight of 1,12-octadecanediol.

2. Compositions according to claim 1, which comprise:
5 to 60 parts by weight of the above-mentioned mixture of alkylpolyglycosides; and
95 to 40 parts by weight of 1,12-octadecanediol.

3. Compositions according to claim 1, wherein the mixture of alkylpolyglycosides mentioned above is constituted of a mixture of any proportion of hydroxyoctadecylpolyglycosides of formula (I) and of polyglycosyloctadecylpolyglycosides of formula (II)
HO—R—O(G)hd n (I)
(G)ₘ—OR—O-13 (G)ₚ (II)
in which:
G represents a saccharide residue;
R represents a 1,12-disubstituted octadecane group
n, m and p represent the average degree of polymerisation of each saccharide residue.

4. Compositions according to claim 2, wherein the mixture of alkylpolyglycosides mentioned above is constituted of a mixture of any proportion of hydroxyoctadecylpolyglycosides of formula (I) and of polyglycosyloctadecylpolyglycosides of formula (II)
HO—R—O(G)ₙ (I)
(G)ₘ—OR—O—(G)ₚ (II)
in which:
G represents a saccharide residue
R represents a 1,12-disubstituted octadecane group;
n, m and p represent the average degree of polymerisation of each saccharide residue.

5. Compositions according to claim 3, wherein in the formulae (I) an d (II) mentioned above:
G represents a glycose residue selected from the group consisting of glucose, dextrose, fructose, galactose, mannose, ribose and xylose; and
n, m and p represent a number between 1.05 and 2.5.

6. Compositions according to claim 4, wherein in the formulae (I) and (II) mentioned above:
G represents a glycose residue selected from the group consisting of glucose, dextrose, fructose, galactose, mannose, ribose and xylose; and
n, m and p represent a number between 1.05 and 2.5.

7. Emulsions comprising an aqueous phase, an oily phase and an emulsifier, wherein said emulsifier is constituted by a composition based on alkylpolyglycosides and fatty diols as defined according to claim 1.

8. Emulsions comprising an aqueous phase, an oily phase and an emulsifier, wherein said emulsifier is constituted by a composition based on alkylpolyglycosides and fatty diols as defined according to claim 3.

9. Emulsions comprising an aqueous phase, an oily phase and an emulsifier, wherein said emulsifier is constituted by a composition based on alkylpolyglycosides and fatty diols as defined according to claim 4.

10. Emulsions comprising an aqueous phase, an oily phase and an emulsifier, wherein said emulsifier is constituted by a composition based on alkylpolyglycosides and fatty diols as defined according to claim 5.

11. Emulsions comprising an aqueous phase, an oily phase and an emulsifier, wherein said emulsifier is constituted by a composition based on alkylpolyglycosides and fatty diols as defined according to claim 6.

12. Emulsions according to claim 11, which comprise:
from 1 to 10% by weight of said composition based on alkylpolyglycosides and fatty diols;
from 0 to 30% by weight of at least one foaming surfactant;
from 0 to 50% by weight of oil; and
an aqueous phase.

13. Emulsions according to claim 11, which comprise
from 1 to 10% by weight of said composition based on alkylpolyglycosides and fatty diols;
from 2 to 30% by weight of at least one foaming surfactant;
from 0 to 50% by weight of oil; and
an aqueous phase.
